(19) United States
(12) Patent Application Publication
(10) Pub. No.: US 2023/0226209 A1
(43) Pub. Date: Jul. 20, 2023

(54) ANTIBODY ENDOHEDRAL METALLOFULLERENE CONJUGATE AND USES THEREOF

(71) Applicant: YAE, LLC, Miami, FL (US)

(72) Inventor: Fernando FISCHMANN, Miami, FL (US)

(73) Assignee: YAE, LLC, Miami, FL (US)

(21) Appl. No.: 18/156,617

(22) Filed: Jan. 19, 2023

Related U.S. Application Data
(60) Provisional application No. 63/300,698, filed on Jan. 19, 2022.

(51) Int. Cl. A61K 47/68; C01B 32/152

(52) U.S. Cl. CPC A61K 47/6803; A61K 47/6851; C01B 32/152

(57) ABSTRACT

The present invention refers to endohedral metallofullerene derivatives for use in the treatment of cancer and other proliferative diseases. In particular, the invention discloses the use of endohedral fullerene antibody conjugates and its derivatives for the treatment of proliferative diseases, in particular, cancer, comprising the combined administration of a metallofullerene-antibody conjugate or a composition comprising the same, and an ionizing radiation therapy, such as, x-ray radiation.

### RELATED APPLICATION

This application claims priority to U.S. Provisional Patent Application No. 63/300,698, filed on Jan. 19, 2022.

### FIELD

Disclosed herein is the use of metallofullerene-antibody conjugates and methods to treat proliferative diseases, particularly cancer. Embodiments include a combined therapy comprising an endohedral metallofullerene-antibody conjugate that targets selectively the cancer cells, and its use for the treatment of cancer in combination with one or more specific ionizing radiation therapies. Embodiments also include a kit comprising such components and method of treatment of proliferative diseases, particularly cancer with said one or more combined therapy.

### BACKGROUND

Normal cells multiply by the highly controlled activation of growth factor receptors by their respective ligands, such as the growth factor receptor tyrosine kinases.

Cancer cells also proliferate by the activation of growth factor receptors, which lose the control of normal proliferation. The loss of control may be caused by a series of factors, such as the autocrine secretion of growth factors, increased expression of receptors, and autonomous activation of biochemical pathways regulated by growth factors.

Normal cells have generally a uniform spheroid cell shape, with a single nucleus, large cytoplasmic volume, a controlled growth and generally remain in their intended location. Malignant cells, such as cancer cells, on the other hand, have irregular cell shapes and sizes, multiple irregular shape nucleuses, small cytoplasmic volumes, and/or have an uncontrolled growth, as depicted, for example, in FIG. 1.

Cancer is the second leading cause of human death next to coronary disease. At a global scale, millions of people die from cancer or related diseases, every year. In the United States alone, as reported by the American Cancer Society, cancer causes the death of more than 500,000 people annually, with over 1.2 million newly diagnosed cases per year. While deaths from heart diseases have been decreasing considerably, those resulting from cancer have reported to be increasing. Cancer is predicted to become the leading cause of death in the upcoming decades.

Worldwide, several malignant tumors stand out as the leading deadly cancers. In particular, carcinomas of the lung, prostate, breast, colon, pancreas, and ovary represent the primary causes of cancer death. These and almost all other carcinomas share a common deadly factor. With very few exceptions, metastatic disease from a carcinoma is fatal. Furthermore, even for those cancer patients who initially survive a first cancer, common experience has shown that their lives are dramatically transformed even if they do not have a new recurring episode. Many cancer patients experience intense metal health consequences driven by the awareness of the potential for recurrence or treatment failure. Many surviving patients experience physical debilitations following treatment, especially those that experience a recurrence.

Among cancers, those that arise from organs and solid tissues, known as solid cancers, such as colon cancer, lung cancer, breast cancer, stomach cancer, prostate cancer, pancreatic cancer, are among the most-commonly identified human cancers.

In the year 2005, an estimated 27 million people were suffering from cancer worldwide and 5.1 million deaths were attributed to malignant tumors. Every year, there are over ten million new cases, a number which is expected to grow by 50% over the next 15 years. Current cancer treatments are generally restricted to invasive surgery, radiation therapy and chemotherapy, each of which is known to be associated with severe side-effects, non-specific toxicity, and/or traumatizing changes to one's body image and/or quality of life. Cancer may become resistant to chemotherapy, which may further reduce options and likelihood of success. The diagnosis for some types of cancer is worse than for others and some, like lung or pancreatic cancer are almost always fatal. In addition, certain cancers with a relatively high treatment success rate, such as breast cancer, also have a very high incidence rate and, thus, remain major killers.

For instance, there are over one million new cases of breast cancer, at a global scale, each year. Treatments to treat breast cancer generally include minimal to radical surgical removal of breast tissue and lymph nodes with radiation and chemotherapy. The success rate for a localized cancer is relatively good with a five years survival rate of around 50%. However, for a metastasized cancer, which is almost incurable, an average survival of around 2 years is expected. Nearly 400,000 women die of breast cancer each year, the highest number of deaths to cancer in woman, ahead of deaths to lung cancer, despite all the improvements in medicine and specific treatments.

Liver cancer represents another cancer with poor prognosis, with more than half a million new cases each year and nearly the same number of deaths due to poor treatment efficacy. Hepatocellular carcinomas are responsible for around 80% of all liver cancers and are rarely curable. Five-year survival rate is only about 10% and survival after diagnosis often less than six months. Although surgical resection of diseased tissue can be effective, it is not an option for the majority of cases because of the presence of cirrhosis of the liver.

The American Cancer Society's estimates for bladder cancer in the United States for 2021 are: about 83,730 new cases of bladder cancer (about 64,280 in men and 19,450 in women) and about 17,200 deaths from bladder cancer (about 12,260 in men and 4,940 in women). The rates of new bladder cancers and deaths linked to bladder cancer and have been dropping slightly in women in recent years. In men, incidence rates have been decreasing, but death rates have been stable. Current treatment includes the intravascular delivery of chemotherapy and immunotherapy with the bacilli Calmette-Guerin (BCG) vaccine that involves the additional risk of systemic infection with the tuberculosis bacterium. Despite this aggressive treatment regime, 70% of these superficial papillary tumors will recur over a prolonged clinical course some will progress into invasive carcinomas.

There are many more examples of malignant tumors where current treatments do not meet the needs of patients either due to their lack of efficacy and/or because they have high morbidity rates and severe side-effects. The statistics and general data presented herein, illustrate well the need for cancer treatments with better safety and efficacy profiles.

One of the causes for the inadequacy of current cancer treatments is their lack of selectivity for affected tissues and cells. Surgical removal generally involves the removal of apparently normal tissue as a “safety margin” which may increase morbidity and risk of complications. It also always removes some of the healthy tissue that may be disseminated with tumor cells and that could potentially maintain or restore the function of the affected organ or tissue. Furthermore, conventional radiation and chemotherapy may kill or damage not only tumor or cancer cells but many normal cells due to their non-specific mode of action. This may result in serious side-effects such as severe nausea, weight loss and hair loss, etc., as well as increasing the risk of developing secondary cancer later in life. Treatment with greater selectivity for cancer cells would leave normal cells unharmed thus improving outcome, side-effect profile and quality of life and is currently one of the biggest challenges in the field.

The selectivity of cancer treatment can be improved by using antibodies that are specific for molecules present only or mostly on cancer cells. Such antibodies can be used to modulate the immune system and enhance the recognition and destruction of the cancer by the patient's own immune system, as well as blocking or altering the function of the target molecule and, thus, of the cancer cells. Antibodies may also be used to target drugs, genes, toxins or other medically relevant molecules to the cancer cells.

### Radiation Therapy

Radiation therapy or radiotherapy is the medical use of ionizing radiation, generally as part of cancer treatment to control or kill malignant cells. Radiation therapy may be curative in a number of types of cancer and may be used as part of curative therapy, to prevent tumor recurrence after surgery to remove a primary malignant tumor. Radiation therapy is generally used before, during, and after chemotherapy in selected cancers.

Radiation therapy is commonly applied to the cancerous tumor because of its ability to control cell growth. Ionizing radiation works by damaging the DNA of exposed tissue leading to cellular death. Normal tissues (such as skin or organs which radiation must pass through in order to treat the tumor), also receive radiation dose causing cellular damage and death which leads to sometimes serious side effects.

Hypoxic tumor cells are resistant to radiation and existing chemotherapy techniques. In contrast to cancerous tumors, normal tissues do not have any hypoxic cells. Accordingly, radiotherapy for treating cancer is more effective when the radiosensitivity of the hypoxic cells in the tumor is enhanced by introducing a radiosensitizer. Attempts have been made to increase the radiosensitivity of cells using different compounds, such as radiosensitizers but the results have been mixed and there are still many challenges to overcome in order to minimize the side effects of radiation therapy while selectively attacking the cancer cell.

In most cases of patients undergoing radiotherapy, doses of radiation on the order of 40-60 Gy to the targeted region, spread throughout different sessions, aimed at producing cell killing are considered to be acceptable or within the medical standards. These “high” doses would decrease with distance from the target tissue, and some tissues might receive doses that are referred to as “low dose” (100 mGy or less).

Diagnostic radiation procedures, in contrast, generally result in small doses to target organs. The following Table summarizes the Estimated Range of Effective Doses from Diagnostic Radiation Exposures

TABLE 1
Range of Effective Doses from Diagnostic Radiation Exposures

| | | Dosage Range |
|---|---|---|
| Procedure | Type of Examination | (mGy)^{†} |
| Conventional simple | Chest films | 0.02-10 mGy |
| X-rays | X-rays of bones and skull | |
| | X-ray of abdomen | |
| Conventional complex | GI series | 3-10 mGy |
| X-rays | Barium enema | |
| | Intravenous urogram | |
| Computed | Head injuries | 5-15 mGy |
| tomography (CT) | Whole-body examinations | |

^{†}The gray (Gy) is a derived unit of ionizing radiation dose in the International System of Units (SI). It is defined as the absorption of one joule of radiation energy per kilogram of matter. It is used as a unit of the radiation quantity absorbed dose that measures the energy deposited by ionizing radiation in a unit mass of matter being irradiated.

In all cases, radiotherapy is known to cause undesirable side effects that at a different extent, may negatively affect the life and recovery of the patient, in particular when high doses need to be administered in a single session or within a short timeframe.

### Computed Tomography

Computed Tomography (CT) has the advantages of high resolution, low cost, and high imaging efficiency, and has become one of the most commonly used imaging methods in clinical practice. CT takes advantage of the difference in absorption and transmittance of X-rays by different tissues of the human body to image the examination site, so as to find subtle lesions in the body. Currently, most CT contrast agents on the market contain iodine compounds. Iodine has a large atomic number, and most of the iodine-containing compounds have strong tissue penetrating power, and enrichment in relevant areas can increase imaging brightness and improve diagnostic accuracy.

However, because these commonly used contrast agents lack affinity with tumor tissue, CT can increase the density of tumor lesions through abundant tumor blood supply, but to a limited extent, and lack specificity for tumor lesions. Therefore, CT and MM scans still lack very reliable parameter indicators for tumor diagnosis.

To this end, one of the purposes and objectives of the present application may also be to provide a tumor-targeted contrast agent which is a venous contrast agent having a tumor tissue guiding function for CT enhanced scanning, and improves the resolution, of tumor lesions by CT scans, and potentially, their treatment.

### Chemical Properties of Fullerenes as Components of the Present Invention

Fullerenes are closed-cage molecules composed entirely of sp2-hybridized carbons, arranged in hexagons and pentagons. Fullerenes (e.g., C60) were first identified as closed spheroidal cages produced by condensation from vaporized carbon. Fullerenes are typically classified according to the number of carbon atoms, e.g., C60-fullerene, C70-fullerene, C76-fullerene, C78-fullerene, C80-fullerene, etc. One of the best-known fullerenes is the Buckminsterfullerene (IUPAC name (C60-Ih) [5,6] fullerene), commonly referred to as “C60” or the “Buckyball”, as depicted in FIG. 2. It will be appreciated that an antibody-endohedral fullerene conjugate may include an endohedral fullerene comprising two or more sp3-hybridized carbons at the point of conjugation.

Fullerenes and their derivatives are useful as superconductor materials, catalysts, and nonlinear optical materials. In the biomedicine field, fullerene compounds find utility as molecular carriers for drugs or catalysts as well as carriers of radioactive metals in targeted therapy for cancer and as a radionuclide tracer.

C60 fullerene is a very strong antioxidant, having an antioxidant capacity of 125 times that of vitamin C. In addition to its antioxidant properties, C60 fullerene also has functions such as scavenging free radicals and activating skin cells (prevention of death). Since 1990, great progress has been made in the research on the free radical scavenging function of C60 fullerenes. Many scientific research results have confirmed that C60 fullerene is a very strong free radical scavenging molecule, which has the potential to bring significant developments in the research of new therapies for the treatment of cancer.

### Endohedral Fullerenes

Endohedral metallofullerenes (EMFs) are fullerenes that encapsulate a metal or a metallic cluster. Endohedral metallofullerenes are an interesting class of fullerenes because electron transfer from the encaged metal atom to the carbon cage has been known to occur, which oftentimes dramatically alters the electronic and magnetic properties of the fullerene. Since the discovery of fullerenes, many attempts to encapsulate different moieties into the carbon structure have resulted in the provision of compounds with remarkable structural, electronic and chemical properties, suitable for many applications in medical and biological diagnostics and therapeutics.

Endohedral metallofullerenes (EMFs) are especially attractive nanoparticles because of their shape, capacity for different encapsulants and excellent isolation from the bio-environment, representing an ideal nanoplatform on which to engineer next-generation diagnostic and therapeutic biomedical agents. These nanoparticles can easily undergo extravasation from the blood pool into tumor tissues and be retained because of poor lymphatic drainage. This phenomenon of selective accumulation of nano-sized particles near tumor tissues is referred to as the enhanced permeability and retention (or EPR) effect, and leads to the accumulation of nanoparticles near tumor tissues. Recent successful manufacturing and isolation of endohedral metallofullerenes has encouraged the chemical functionalization of endohedral metallofullerenes, which facilitates the clarification of their physical and chemical properties.

The therapeutic potential of EMFs has been an area of great interest as well. Numerous studies have demonstrated the free radical scavenging capabilities of fullerenes, to such a degree that some authors have described them as “free radical sponges.” In addition, the antineoplastic and radical scavenging properties for a few metallofullerenes, such as the Gd@C82 platform have been reported.

Due to the severity and breadth of proliferative diseases, including tumor and cancer, there is a great need for effective pharmaceutical compositions, uses and methods for the treatment of such diseases that overcome the shortcomings of surgery, chemotherapy, and radiation treatment. Fullerenes, and in particular, the endohedral fullerenes described herein, represent suitable candidates for the development of successful novel methods for the treatment of proliferative diseases, including cancer.

### BRIEF DESCRIPTION OF THE FIGURES

FIG. 1 shows a schematic representation of normal cells (1) versus cancer or malignant (2) cells.

FIG. 2 shows a schematic representation of the fullerene C60-cage chemical structure.

FIG. 3. shows a schematic representation of an endohedral fullerene submitted to ionizing radiation.

FIG. 4 shows a schematic representation of a normal cell (1), a normal membrane receptor (1a), a malignant cell (2), altered membrane receptors (3), and the binding of the altered membrane receptor (3) to the metallofullerene-antibody conjugate (5).

FIG. 5 shows a schematic representation the binding of the altered membrane receptor (3) to the metallofullerene-antibody conjugate (5), which are subjected to low level ionizing radiation (6).

FIG. 6 shows a schematic representation the binding of the altered membrane receptor (3) to a cell binding moiety (4) of the metallofullerene-antibody conjugate (5) comprising two fullerene moieties, which are subjected to low level radiation (6).

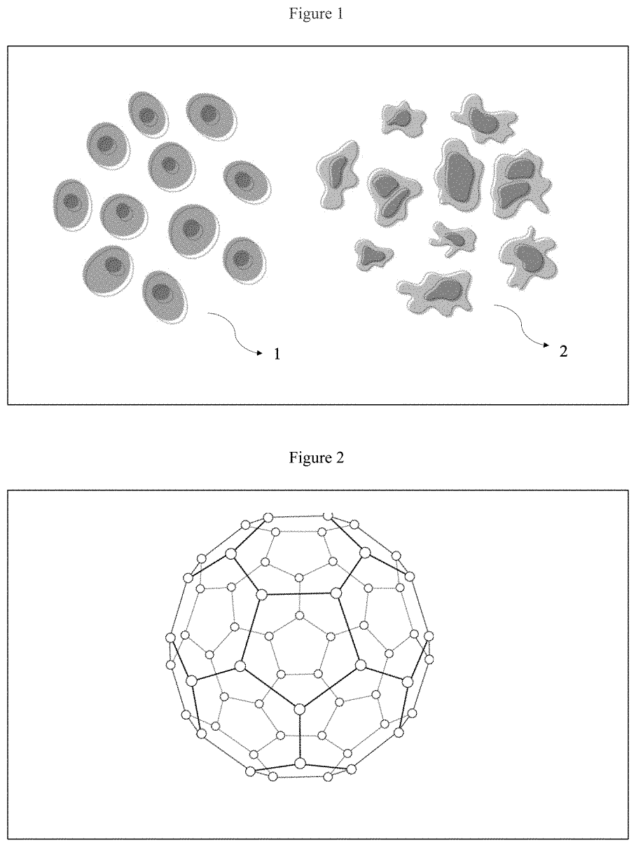

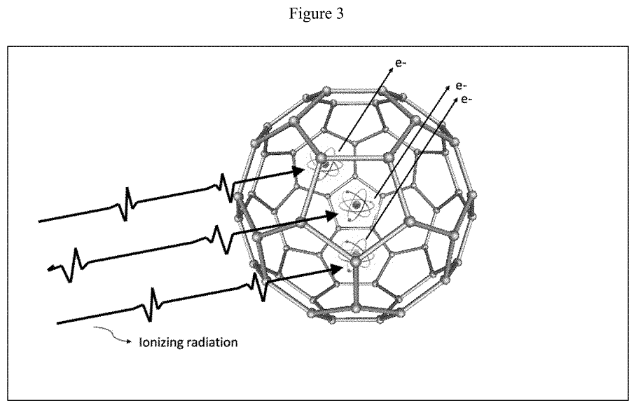

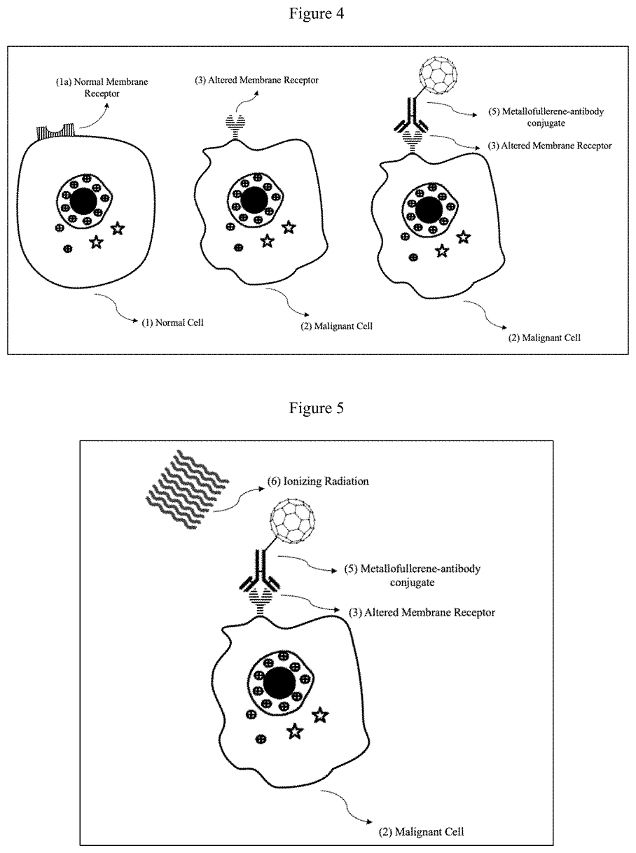

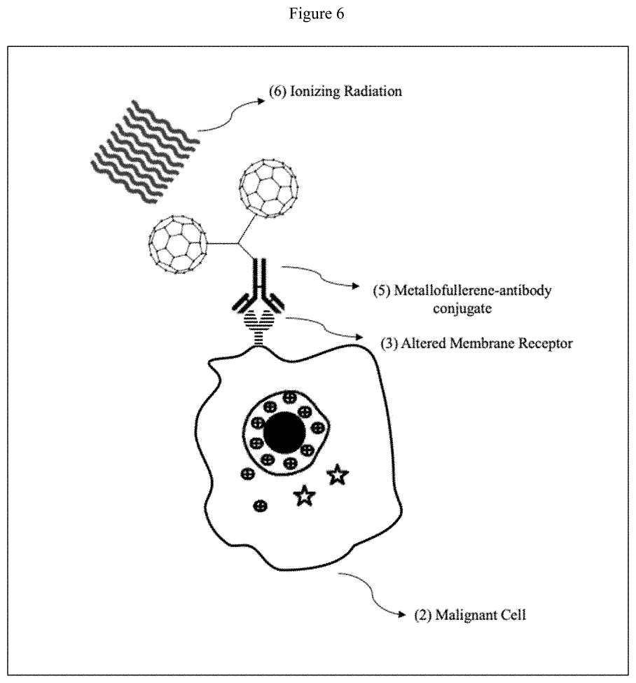

### DETAILED DESCRIPTION

Reference now will be made in detail to certain embodiments. While enumerated embodiments will be described, it will be understood that they are not intended to limit the invention to those embodiments. On the contrary, the invention is intended to cover alternatives, modifications, and equivalents, which may be included within the scope of the present invention as defined by the claims. The skilled artisan will recognize several methods equivalent to those described herein, which could be used in the practice of the present invention. In the event that one or more of the incorporated literature and similar materials differs from or contradicts this application, including but not limited to defined terms, term usage, described techniques, or the like, this application controls.

In order to solve the above-mentioned problems in the prior art, the purpose of the present disclosure is to provide endohedral metallofullerene derivatives therapeutically activated by ionizing radiation for use in the treatment of proliferative diseases, including cancer, as well as a therapeutic method for the treatment of tumors and cancers.

The present invention is based on the discovery that administering to the patient a monoclonal or polyclonal antibody-endohedral metallofullerene conjugate, or a composition comprising the same, targeted to the altered receptor or membrane of a specific cancer cell, wherein the metal is a rare-earth element, and subsequently submitting the patient, and particularly the area where the tumor is located, with a source of a low intensity ionizing radiation such as an x-ray radiation, it is possible to target said cancer cells in situ with great treatment efficacy, damaging the cell's genetic material, its DNA, its membranes, receptors, or a combination thereof requiring less dose of radiation and subsequently causing lower to none undesirable side effects.

In particular, it has been surprisingly found that the reactivity of the rare-earth metal element to the ionizing radiation may be exponentially increased by embedding the metal element within the fullerene, forming thus an endohedral fullerene. The reactivity of a fullerene, such as a 60-, a 80-carbon, or a 82-carbon fullerene derivative enclosing a rare-earth lanthanide metal, to x-ray radiation, has shown promising results. Lanthanide endohedral fullerenes, when subjected to an x-ray radiation of about e.g., 160 electron volts, a reaction is developed that triggers the lanthanide element to lose an electron, which transitions to a lower level of the atom. The vacant spot of that transitioning electron is subsequently occupied by an outer electron from the rare-earth lanthanide element and the energy created by such electron transition is absorbed by the carbon fullerene cage or another of the lanthanide atoms. This phenomenon, also referred hereby to as an electron transfer chain reaction, facilitates the induction of radiation cell damage by submitting the molecule to a low x-ray radiation, which based on the energy absorption that the fullerene cage creates, may enhance the effect of such low x-radiation creating a highly specific localized and targeted cellular damage. Furthermore, the electron transfer chain reaction described herein enables an in situ cellular damage in all malignant cells that surround, or are located in the vicinity of the metallofullerene-antibody conjugate, which increases the selectivity of the treatment and reduces damage to healthy normal cells. The metallofullerene-antibody conjugate of the present invention has therefore the potential of acting as an acceptor of ionizing radiation as well as enhancer of the same.

The present invention provides an endohedral metallofullerene-antibody conjugate for use in the treatment of proliferative diseases, in particular, cancer, as well as compositions comprising said conjugates. The present invention further describes a method that comprises administering an endohedral metallofullerene-antibody conjugate to a patient in combination with a low-level ionizing radiation, such as x-ray radiation. The endohedral metallofullerene-antibody conjugate and method of the invention have surprisingly shown to limit the need of exposure of said radiation in patients while achieving high treatment efficiency. In another embodiment, the present invention provides a composition comprising an endohedral metallofullerene and its use as tumor-targeting contrast agent.

The present invention represents a breakthrough development in the state-of-the-art therapies for cancer and other proliferative diseases, with advantages that include:

Highly Specific Targeting of Malignant (Cancer) Cells

The in situ cellular damage in cancer cells achieved by the present invention is facilitated due to the administration of the metallofullerene-antibody conjugate that targets specific malignant cells and accumulates in their immediate vicinity. As explained above, the metallofullerene-antibody conjugate acts both as an acceptor of ionizing radiation as well as enhancer of the same and such targeted administration of the metallofullerene-antibody conjugate increases the specificity of the ionizing radiation that targets and kills malignant cells only, reducing thus the damage to healthy normal cells, in sharp contrast with what it is normally seen in conventional radiotherapy.

Lower Levels of Ionizing Radiation

As a result of the increased specificity of the method of the present invention, the radiation dose can be drastically decreased while maintaining, or even improving the effect of the ionizing radiation, while at the same time lowering its radiotoxicity. As it will be explained in the detailed description, the present invention has the potential of reducing the amount of ionizing radiation necessary to destroy a malignant cell, as compared to conventional treatments

Wide Spectrum of Cancer Cell Types

Given that the endohedral metallofullerene-antibody conjugate of the present invention may include one or more antibody carriers that bind to different growth factors, receptors or membrane, the method of the invention has the capacity of targeting and killing a wide range of malignant cancers, including lung cancer, prostate cancer, head and neck cancer, pancreatic cancer, colon/colorectal cancer, bladder cancer, thyroid cancer, breast cancer, liver cancer, ovarian cancer, endometrial cancer, cervical cancer cells, kidney cancer, brain cancer, or melanoma cells. In some cases, the tumor cells can be non-small cell lung cancer (NSCLC) cells.

In a particular embodiment of the invention, a metallofullerene-antibody conjugate wherein the fullerene is an endohedral metal fullerene is described, which is useful in the treatment of proliferative diseases, including cancer.

According to the invention, the endohedral metallofullerene-antibody conjugate is represented by formula (I)

AbCₘMₙ@F₂ᵥ (I)

for use in the treatment of proliferative diseases in combination with a low-level ionizing radiation wherein the metallofullerene-antibody conjugate is therapeutically activated by the ionizing radiation,

wherein Ab represents a cell binding agent;

wherein M represents a rare earth element in the period table of elements selected from scandium, yttrium, lanthanum, cerium, praseodymium, neodymium, promethium, samarium, europium, gadolinium, terbium, dysprosium, holmium, erbium, thulium, ytterbium, lutetium, or combinations thereof;

wherein F represents a fullerene having v carbon atoms;

wherein C represents a linker chemically bonding the fullerene F with the antibody A;

wherein n represents an integer from 1 to 3;

wherein m represents an integer from 0 to 3; and

wherein v represents an integer from about 20 to about 60.

In one aspect of the invention, Ab is a cell binding agent selected from a chimeric, CDR-grafted, humanized, or recombinant human antibody. Preferably, Ab binds specifically to a tumor-associated antigen or cell-surface receptor. In another aspect, Ab is selected from the group of antibodies or antibody fragments used in cancerology, targeting: HER2, CD 52, VEGF (vascular endothelial growth factor), EGF R (epidermal growth factor receptor), CD11a, CCR4 (chemokine C-C receptor 4), CD 105, CD 123, CD 137, CD 19, CD 22, CD 23, CD 3, CD 30, CD 38, CD 4, CD 40, CD 55SC-1, CD 56, CD 6, CD 74, CD 80, CS 1 (cell-surface glycoprotein 1), CTLA4 (cytotoxic T-lymphocyte antigen 4, also known as CD152), DR5 (death receptor 5), Ep-CAM (epithelial cell adhesion molecule), folate receptor alpha, ganglioside GD2, ganglioside GD3, GPNMB, glycoprotein NMB, HGF/SF (hepatocyte growth factor/scatter factor), IGF-1 (insulin-like growth factor), IGF1-receptor (insulin-like growth factor-1 receptor), IL 13 (interleukin-13), IL 6 (interleukin-6), IL-6R (interleukin-6 receptor), immunodominant fungal antigen heat shock protein 90 (hsp90), integrin alpha 5 beta 3, MHC (major histocompatibility complex) class II, MN-antigen (G250 antigen), MUC1, PD-1 (programmed death 1), PIGF (placental growth factor), PDGFRa (platelet-derived growth factor receptor alpha), prostate specific membrane antigen (PSMA), PTHrP (parathyroid hormone-related protein), CD200 receptor, receptor activator of nuclear factor kappa B ligand (RANKL), sphingosine-1-phosphate (SIP), TGF beta, TRAIL (tumor necrosis factor (TNF)-related apoptosis-inducing ligand) receptor 1, tumor necrosis factor receptor 2, vascular endothelial growth factor receptor 2 (VEGFR-2), CD 33, CD 20, CA125 (cancer antigen 125) or epidermal growth factor receptor.

In another aspect, the antibody of the metallofullerene-antibody conjugate is selected from HER2 (Trastuzumab)—a therapeutic monoclonal antibody specific for the human epidermal growth factor receptor type 2 (HER2), a cell-surface tyrosine kinase receptor overexpressed by 25% to 30% of breast cancers. In another aspect, the antibody of the metallofullerene-antibody conjugate is selected from EGFR (Depatuxizumab)—a monoclonal antibody specific to the Epidermal growth factor receptor, whose overexpression is associated to a number of cancers overexpression (known as upregulation or amplification) have been associated with a number of cancers, including adenocarcinoma of the lung (40% of cases), anal cancers, glioblastoma (50%) and epithelium tumors of the head and neck. In another aspect, the antibody of the metallofullerene-antibody conjugate is selected from VEGF (Varisacumab)—an antibody specific to the Vascular endothelial growth factor whose overexpression may be an early step in the process of metastasis.

In another aspect, M preferably represents a rare-earth element in the period table of elements selected from Holmium, Lutetium, Scandium, Gadolinium and combinations thereof.

In one aspect, the antibody may comprise multiple endohedral metallofullerenes, such as, two, or three, to provide a conjugate represented as, for example, Ab[CMₙ@F₂ᵥ,CMₙ@₂ᵥ], when m is 2, for example.

In another aspect, m is 0 (zero) and in that case, the Ab and the M moieties are linked directly.

Endohedral metallofullerenes of Formula (I) and their respective antibody conjugates may prepared according to known procedures. See, e.g., Bolskar, Chen, Shultz, and Yang.

In still another aspect, the invention provides pharmaceutical compositions comprising an effective amount of the metallofullerene-antibody conjugate of Formula I and a pharmaceutically acceptable carrier or vehicle for use in the treatment of proliferative diseases in combination with a low-level ionizing radiation. Preferably, for the use in the treatment of proliferative diseases in combination with a low-level ionizing radiation, wherein the metallofullerene-antibody conjugate is therapeutically activated by ionizing radiation.

In still another aspect, the invention provides the use of the endohedral metallofullerene-antibody conjugate for the treatment of a proliferation-related disorder or disease. The invention also provides the use of the metallofullerene-antibody conjugate, in combination with a low-level ionizing radiation for the treatment of a proliferation related disorder or disease. In still another aspect, the invention provides the use of a metallofullerene-antibody conjugate which is therapeutically activated through ionizing radiation, such as x-ray, for the treatment of a proliferation related disorder or disease.

In another aspect, the invention includes a method of treating cancer comprising administering to a human with a hyperproliferative disorder, a composition comprising a metallofullerene-antibody conjugate and a pharmaceutically acceptable diluent, carrier or excipient. In another aspect, the invention provides methods for preventing and/or treating the multiplication of a tumor cell or cancer cell including administering to a mammal, such as a patient with a hyperproliferative disorder, an effective amount of a metallofullerene-antibody conjugate in combination with a low-level ionizing radiation for the treatment of a proliferation related disorder or disease, preferably cancer.

In another aspect, the invention includes a pharmaceutical composition comprising an effective amount of an endohedral metallofullerene-antibody conjugate of Formula I, or a pharmaceutically acceptable salt thereof, and a pharmaceutically acceptable diluent, carrier or excipient. The composition may further comprise a therapeutically effective amount of chemotherapeutic agent and is provided in combination with a low-level ionizing radiation for the treatment of a proliferation related disorder or disease.

The pharmaceutical composition is in a form suitable for oral administration, intravenous administration, topical administration, administration by inhalation, or administration via a suppository.

A method according to the invention may comprise a step of providing a tumor area with an endohedral metallofullerene-antibody conjugate of Formula I, or a composition comprising the conjugate and exposing the processed tumor area to ionizing radiation as defined herein.

A method according to the invention may also comprise a step of processing a tumor area with a metallofullerene-antibody conjugate of Formula I, allowing the conjugate or the components thereof to concentrate in or around the tumor area, and exposing the processed tumor area to ionizing radiation as defined herein.

The administration of a metallofullerene-antibody conjugate of Formula I and ionizing radiation, preferably a low intensity radiation has shown to produce synergic effects, such as anti-tumor effects, greater than those combined effects achieved with any of the metallofullerene-antibody conjugate of Formula I, its moieties and ionizing radiation individually.

In a further aspect, the present invention discloses the use of an endohedral metallofullerene-antibody conjugate of Formula I for use in the preparation of CT contrast media.

In the present invention, the term “ionizing radiation” denotes radiation comprising particles or photons that have sufficient energy or can produce sufficient energy via nuclear interactions to produce ionization (gain or loss of electrons). An exemplary and preferred ionizing radiation is an x-radiation, which is preferably a low intensity radiation. Means for delivering x-radiation to a target tissue or cell are well known in the art. The amount of ionizing radiation needed in a given cell generally depends on the nature of that cell but are generally lower than the ones required by traditional radiotherapy. Used herein, the term “an effective dose” of ionizing radiation means a dose of ionizing radiation that produces an increase in cell damage or death when given in conjunction with the endohedral fullerene conjugates of the invention, optionally further combined with a chemotherapeutic agent. An effective dose may include, but is not limited to a dose of from about 0.02 mGy to about 60 Gy, and all values in between, including, for example, 0.04 mGy, 0.06 mGy, 0.08 mGy, 0.1 mGy, 0.2 mGy, 0.4 mGy, 0.6 mGy, 0.8 mGy, 1 mGy, 2 mGy, 4 mGy, 6 mGy, 8 mGy, 10 mGy, 20 mGy, 40 mGy, 60 mGy, 80 mGy, 100 mGy, 200 mGy, 400 mGy, 600 mGy, 800 mGy, 1000 mGy (or 1 Gy), 2 Gy, 4 Gy, 6 Gy, 8 Gy, 10 Gy, 12, Gy, 14, Gy, 16 Gy, 18 Gy, 20 Gy, 22 Gy, 24 Gy, 26 Gy, 28 Gy, 30 Gy, 32 Gy, 34 Gy, 36 Gy, 38 Gy, 40 Gy, 42 Gy, 44 Gy, 46 Gy, 48 Gy, 50 Gy, 52 Gy, 54 Gy, 56 Gy, and 58 Gy.

The phrase “pharmaceutically acceptable” indicates that the substance or composition is compatible chemically and/or toxicologically, with the other ingredients comprising a formulation, and/or the mammal being treated therewith.

The phrases “therapeutically effective amount” or “effective amount” mean an amount of a compound described herein that, when administered to a mammal in need of such treatment, is sufficient to (i) treat or prevent the particular disease, condition, or disorder, (ii) attenuate, ameliorate, or eliminate one or more symptoms of the particular disease, condition, or disorder, or (iii) prevent or delay the onset of one or more symptoms of the particular disease, condition, or disorder described herein. The amount of a compound that will correspond to such an amount will vary depending upon factors such as the particular compound, disease condition and its severity, the identity (e.g., weight) of the person in need of treatment, but can nevertheless be routinely determined by one skilled in the art.

The terms “treat” or “treatment” refer to therapeutic, prophylactic, palliative or preventative measures. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder, as well as those prone to have the condition or disorder or those in which the condition or disorder is to be prevented.

One may determine the amount of endohedral metallofullerene-antibody conjugate to be used in a given treatment (e.g., treatment of a proliferative disorder (e.g., cancer) or CT scanning). For instance, HERCEPTIN® (trastuzumab) for injection is indicted generally for the treatment of adjuvant breast cancer, metastatic breast cancer, and metastatic breast cancer according to a certain dosing amount and schedule. HERCEPTIN® PI. An amount of an endohedral metallofullerene-antibody conjugate disclosed herein may be dosed at the same amount, as for HERCEPTIN®, e.g., 2 mg/kg, 4 mg/kg, 6 mg/kg, and/or 8 mg/kg. The dosing schedule may be associated with the administration of a low-level ionizing radiation as disclosed herein in a manner to minimize tumor growth and/or size. In view of the foregoing, an effective amount of endohedral metallofullerene-antibody conjugate ranges from about 0.001 mg/kg to about 100 mg/kg and all values in between.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

### Aspects

Aspect 1. An endohedral metallofullerene-antibody conjugate of formula (I)

AbCₘMₙ@F₂ᵥ (I)
wherein Ab represents a cell binding agent;
wherein M represents a rare earth element in the period table of elements selected from the group comprising scandium, yttrium, lanthanum, cerium, praseodymium, neodymium, promethium, samarium, europium, gadolinium, terbium, dysprosium, holmium, erbium, thulium, ytterbium, and lutetium, or combinations thereof;
wherein F represents a fullerene molecule having v carbon atoms;
wherein C represents a linker connecting the fullerene F with the Ab;
wherein n represents an integer from 1 to 3;
wherein m represents an integer from 0 to 3; and
wherein v represents an integer from about 20 to about 60;
for use in the treatment of proliferative diseases in combination with a low-level ionizing radiation, wherein the metallofullerene-antibody conjugate is optionally therapeutically activated by the ionizing radiation.

Aspect 2. The metallofullerene-antibody conjugate of Aspect 1, wherein Ab is a cell binding agent selected from the group comprising a chimeric, CDR-grafted, humanized, or recombinant human antibody, more preferably, Ab binds specifically to a tumor-associated antigen or cell-surface receptor.

Aspect 3. The metallofullerene-antibody conjugate of any one of Aspects 1-2, wherein Ab is selected from the group of antibodies or antibody fragments used in cancerology, targeting: HER2, HER2, HER3, HER4 CD 52, VEGF (vascular endothelial growth factor), EGFR (epidermal growth factor receptor), CD11a, CCR4 (chemokine C-C receptor 4), CD 105, CD 123, CD 137, CD 19, CD 22, CD 23, CD 3, CD 30, CD 38, CD 4, CD 40, CD 55SC-1, CD 56, CD 6, CD 74, CD 80, CS 1 (cell-surface glycoprotein 1), CTLA4 (cytotoxic T-lymphocyte antigen 4, also known as CD152), DR5 (death receptor 5), Ep-CAM (epithelial cell adhesion molecule), folate receptor alpha, ganglioside GD2, ganglioside GD3, GPNMB, glycoprotein NMB, HGF/SF (hepatocyte growth factor/scatter factor), IGF-1 (insulin-like growth factor), IGF1-receptor (insulin-like growth factor-1 receptor), IL 13 (interleukin-13), IL 6 (interleukin-6), IL-6R (interleukin-6 receptor), immunodominant fungal antigen heat shock protein 90 (hsp90), integrin alpha 5 beta 3, MHC (major histocompatibility complex) class II, MN-antigen (also known as G250 antigen), MUC1, PD-1 (programmed death 1), PIGF (placental growth factor), PDGFRa (platelet-derived growth factor receptor alpha), prostate specific membrane antigen (PSMA), PTHrP (parathyroid hormone-related protein), CD200 receptor, receptor activator of nuclear factor kappa B ligand (RANKL), sphingosine-1-phosphate (SIP), TGF beta (transforming growth factor beta), TRAIL (tumor necrosis factor (TNF)-related apoptosis-inducing ligand) receptor 1, tumor necrosis factor receptor 2, vascular endothelial growth factor receptor 2 (VEGFR-2), CD 33, CD 20, CA125 (cancer antigen 125) or epidermal growth factor receptor.

Aspect 4. The metallofullerene-antibody conjugate of Aspect 2, wherein the antibody is selected from one or more of EGFR, HER2, HER3, and HER4 binding antibody.

Aspect 5. The metallofullerene-antibody conjugate of Aspect 2, wherein the antibody is selected from one or more of Depatuxizumab, Varisacumab, and Trastuzumab.

Aspect 6. The metallofullerene-antibody conjugate of any one of Aspects 1-5, wherein M is selected from one or more of Holmium, Lutetium, and Scandium.

Aspect 7. The metallofullerene-antibody conjugate of any one of Aspects 1-6, wherein the proliferative disease is cancer.

Aspect 8. The metallofullerene-antibody conjugate of Aspect 7, wherein the type of cancer is selected from one or more of lung cancer, prostate cancer, head and neck cancer, pancreatic cancer, colon/colorectal cancer, bladder cancer, thyroid cancer, breast cancer cells, liver cancer, ovarian cancer, endometrial cancer, cervical cancer cells kidney cancer, brain cancer, and melanoma.

Aspect 9. The metallofullerene-antibody conjugate of any one of Aspects 1-8, wherein the low-level ionizing radiation ranges from about 0.02 mGy to about 60 Gy.

Aspect 10. A pharmaceutical composition comprising the metallofullerene-antibody conjugate of any one of Aspects 1-8, and a pharmaceutically acceptable excipient, diluent, or vehicle.

Aspect 11. The composition of Aspect 10, in a form suitable for oral administration, intravenous administration, topical administration, administration by inhalation, or administration via a suppository.

Aspect 12. The metallofullerene-antibody conjugate of any one of Aspects 1-9 or the pharmaceutical composition of any one of Aspects 10-11, further comprising an additional therapeutic agent.

Aspect 13. A method of treating a cell proliferation related disorder or disease, comprising
administering to a patient an effective amount of a metallofullerene-antibody conjugate of Formula I

AbCₘMₙ@F₂ᵥ (I)
wherein Ab represents a cell binding agent;
wherein M represents a rare earth element in the period table of elements selected from scandium, yttrium, lanthanum, cerium, praseodymium, neodymium, promethium, samarium, europium, gadolinium, terbium, dysprosium, holmium, erbium, thulium, ytterbium, and lutetium, or combinations thereof;
wherein F represents a fullerene molecule having v carbon atoms;
wherein C represents a linker connecting the fullerene F with the Ab;
wherein n represents an integer from 1 to 3;
wherein m represents an integer from 0 to 3; and
wherein v represents an integer from about 20 to about 60; and
administering a low-level ionizing radiation, wherein the metallofullerene-antibody conjugate is preferably therapeutically activated by the ionizing radiation.

Aspect 14. The method of Aspect 13, wherein Ab is a cell binding agent selected from the group comprising a chimeric, CDR-grafted, humanized, or recombinant human antibody, more preferably, Ab binds specifically to a tumor-associated antigen or cell-surface receptor.

Aspect 15. The method of any one of Aspects 13-14, wherein Ab is selected from the group of antibodies or antibody fragments used in cancerology, targeting: HER2, HER2, HER3, HER4 CD 52, VEGF (vascular endothelial growth factor), EGFR (epidermal growth factor receptor), CD11a, CCR4 (chemokine C-C receptor 4), CD 105, CD 123, CD 137, CD 19, CD 22, CD 23, CD 3, CD 30, CD 38, CD 4, CD 40, CD 55SC-1, CD 56, CD 6, CD 74, CD 80, CS 1 (cell-surface glycoprotein 1), CTLA4 (cytotoxic T-lymphocyte antigen 4, also known as CD152), DR5 (death receptor 5), Ep-CAM (epithelial cell adhesion molecule), folate receptor alpha, ganglioside GD2, ganglioside GD3, GPNMB, glycoprotein NMB, HGF/SF (hepatocyte growth factor/scatter factor), IGF-1 (insulin-like growth factor), IGF1-receptor (insulin-like growth factor-1 receptor), IL 13 (interleukin-13), IL 6 (interleukin-6), IL-6R (interleukin-6 receptor), immunodominant fungal antigen heat shock protein 90 (hsp90), integrin alpha 5 beta 3, MHC (major histocompatibility complex) class II, MN-antigen (also known as G250 antigen), MUC1, PD-1 (programmed death 1), PIGF (placental growth factor), PDGFRa (platelet-derived growth factor receptor alpha), prostate specific membrane antigen (PSMA), PTHrP (parathyroid hormone-related protein), CD200 receptor, receptor activator of nuclear factor kappa B ligand (RANKL), sphingosine-1-phosphate (SIP), TGF beta (transforming growth factor beta), TRAIL (tumor necrosis factor (TNF)-related apoptosis-inducing ligand) receptor 1, tumor necrosis factor receptor 2, vascular endothelial growth factor receptor 2 (VEGFR-2), CD 33, CD 20, CA125 (cancer antigen 125) or epidermal growth factor receptor.

Aspect 16. The method of Aspect 14, wherein the antibody is selected from one or more of EGFR, HER2, HER3, and HER4 binding antibody.

Aspect 17. The method of Aspect 14, wherein the antibody is selected from one or more of Depatuxizumab, Varisacumab, and Trastuzumab.

Aspect 18. The method of any one of Aspects 13-17, wherein M is selected from one or more of Holmium, Lutetium, and Scandium.

Aspect 19. The method of any one of Aspects 13-18, wherein the proliferative disease is cancer.

Aspect 20. The method of Aspect 19, wherein the type of cancer is selected from one or more of lung cancer, prostate cancer, head and neck cancer, pancreatic cancer, colon/colorectal cancer, bladder cancer, thyroid cancer, breast cancer cells, liver cancer, ovarian cancer, endometrial cancer, cervical cancer cells, kidney cancer, brain cancer, and melanoma.

Aspect 21. The method of any one of Aspects 13-20, wherein the low-level ionizing radiation ranges from about 0.02 mGy to about 60 Gy.

Aspect 22. The method of any one of Aspects 13-21, wherein the conjugate is comprised in a pharmaceutically acceptable composition comprising an excipient, diluent, or vehicle.

Aspect 23. The method of Aspect 22, wherein the pharmaceutical composition is in a form suitable for oral administration, intravenous administration, topical administration, administration by inhalation, or administration via a suppository.

Aspect 24. The method of any one of Aspects 13-23, further comprising administering one or more of an additional therapeutic agent.

Aspect 25. The method of any one of Aspects 13-24, wherein the metallofullerene-antibody conjugate is administered in a dose of an intravenous infusion.

Aspect 26. The method of any one of Aspects 13-25, wherein said conjugate is administered to the patient as a first line therapy.

Aspect 27. The method of any one of Aspects 13-25, wherein said conjugate is administered to the patient as a second, third, fourth, fifth, or sixth line of treatment.

Aspect 28. The method of any one of Aspects 13-27, wherein said conjugate is administered to the patient following treatment with at least one anti-cancer therapy.

Aspect 29. The method of any one of Aspects 13-28, wherein said cell proliferation related disorder or disease is resistant to at least one anti-cancer agent.

Aspect 30. The method of any one of Aspects 13-29, wherein said method of treating a cell proliferation related disorder or disease inhibits metastasis in said patient.

Aspect 31. The method of any one of Aspects 13-30, wherein said conjugate is administered to the patient and subsequently administering a low-level ionizing radiation once the conjugate has been concentrated on the target cell.

Aspect 32. The method of any one of Aspects 13-31, wherein the low-level ionizing radiation is administered to the patient within 24 hours, preferably 12 hours, and more preferably within six hours upon the administration of the conjugate.

Aspect 33. An endohedral metallofullerene-antibody conjugate for use in the preparation of computed tomography contrast media of formula (I)

AbCₘMₙ@F₂ᵥ (I)
wherein Ab represents a cell binding agent;
wherein M represents a rare earth element in the period table of elements selected from scandium, yttrium, lanthanum, cerium, praseodymium, neodymium, promethium, samarium, europium, gadolinium, terbium, dysprosium, holmium, erbium, thulium, ytterbium, and lutetium, or combinations thereof;
wherein F represents a fullerene molecule having v carbon atoms;
wherein C represents a linker connecting the fullerene F with the Ab;
wherein n represents an integer from 1 to 3;
wherein m represents an integer from 0 to 3; and
wherein v represents an integer from about 20 to about 60.

Aspects 2-12 are likewise applicable to Aspect 33. For instance, Aspect 2 specifies that Ab is a cell binding agent selected from the group comprising a chimeric, CDR-grafted, humanized, or recombinant human antibody, more preferably, Ab binds specifically to a tumor-associated antigen or cell-surface receptor. Aspect 2 is likewise applicable to Aspect 33, as well as any one of Aspects 3-12.

Aspect 34. A method of imaging a tumor in a patient, said method comprises:
administering to the patient an effective amount of endohedral metallofullerene-antibody conjugate of Aspect 33 and
exposing a tissue of the patient to a low-level ionizing radiation.

Aspects 2-12 are likewise applicable to Aspect 34. For instance, Aspect 2 specifies that Ab is a cell binding agent selected from the group comprising a chimeric, CDR-grafted, humanized, or recombinant human antibody, more preferably, Ab binds specifically to a tumor-associated antigen or cell-surface receptor. Aspect 2 is likewise applicable to Aspect 34, as well as any one of Aspects 3-12.

### DISCLOSED INFORMATION

Chen et al., Applications of functionalized fullerenes in tumor theranostics, Theranostics (2012) 2: 238-250 (“Chen”).
HERCEPTIN® (trastuzumab) for injection prescribing information, as of Nov. 29, 2018 (“HERCEPTIN® PI”).
Shultz et al., Encapsulation of a radiolabeled cluster inside a fullerene cage ¹⁷⁷LuₓLu₍₃₋ₓ₎N@C₈₀: an Interleukin-13-conjugated radiolabeled metallofullerene platform, J. Am. Chem. Soc. (2010) 132(14): 4980-4981.
U.S. Pat. No. 7,208,132 B2, Purification of endohedral and other fullerenes by chemical methods, issued on Apr. 24, 2007 to Bolskar et al. of TDA Research, Inc. and U.S. Pat. No. 7,671,230 B2, Bolskar et al., Derivation and solubilization of insoluble classes of fullerenes, issued on Mar. 2, 2010 to Bolskar et al. of TDA Research, Inc. (collectively “Bolskar”).
Yang et al., Fullerene-biomolecule conjugates and their biomedical applications, International Journal of Nanomedicine (2014) 9:77-92 (“Yang”).

The subject matter of U.S. Provisional Patent Application No. 63/300,698, filed on Jan. 19, 2022, is incorporated by reference. To the extent necessary, information disclosed herein is hereby incorporated by reference. If incorporated subject matter conflicts with subject matter disclosed herein, the subject matter disclosed herein controls.

1. An endohedral metallofullerene-antibody conjugate of formula (I)
AbCₘMₙ@F₂ᵥ (I)
wherein Ab represents a cell binding agent;
wherein M represents a rare earth element in the period table of elements selected from the group comprising scandium, yttrium, lanthanum, cerium, praseodymium, neodymium, promethium, samarium, europium, gadolinium, terbium, dysprosium, holmium, erbium, thulium, ytterbium, and lutetium, or combinations thereof;
wherein F represents a fullerene molecule having v carbon atoms;
wherein C represents a linker connecting the fullerene F with the Ab;
wherein n represents an integer from 1 to 3;
wherein m represents an integer from 0 to 3; and
wherein v represents an integer from about 20 to about 60;
wherein the metallofullerene-antibody conjugate is optionally therapeutically activated by the ionizing radiation.

2. The metallofullerene-antibody conjugate of claim 1, wherein Ab is a cell binding agent selected from the group comprising a chimeric, CDR-grafted, humanized, or recombinant human antibody.

3. The metallofullerene-antibody conjugate of claim 1, wherein Ab is selected from the group of antibodies or antibody fragments used in cancerology, targeting: HER2, HER2, HER3, HER4 CD 52, VEGF (vascular endothelial growth factor), EGFR (epidermal growth factor receptor), CD11a, CCR4 (chemokine C-C receptor 4), CD 105, CD 123, CD 137, CD 19, CD 22, CD 23, CD 3, CD 30, CD 38, CD 4, CD 40, CD 55SC-1, CD 56, CD 6, CD 74, CD 80, CS 1 (cell-surface glycoprotein 1), CTLA4 (cytotoxic T-lymphocyte antigen 4, also known as CD152), DR5 (death receptor 5), Ep-CAM (epithelial cell adhesion molecule), folate receptor alpha, ganglioside GD2, ganglioside GD3, GPNMB, glycoprotein NMB, HGF/SF (hepatocyte growth factor/scatter factor), IGF-1 (insulin-like growth factor), IGF1-receptor (insulin-like growth factor-1 receptor), IL 13 (interleukin-13), IL 6 (interleukin-6), IL-6R (interleukin-6 receptor), immunodominant fungal antigen heat shock protein 90 (hsp90), integrin alpha 5 beta 3, MHC (major histocompatibility complex) class II, MN-antigen (also known as G250 antigen), MUC1, PD-1 (programmed death 1), PIGF (placental growth factor), PDGFRa (platelet-derived growth factor receptor alpha), prostate specific membrane antigen (PSMA), PTHrP (parathyroid hormone-related protein), CD200 receptor, receptor activator of nuclear factor kappa B ligand (RANKL), sphingosine-1-phosphate (SIP), TGF beta (transforming growth factor beta), TRAIL (tumor necrosis factor (TNF)-related apoptosis-inducing ligand) receptor 1, tumor necrosis factor receptor 2, vascular endothelial growth factor receptor 2 (VEGFR-2), CD 33, CD 20, CA125 (cancer antigen 125) or epidermal growth factor receptor.

4. The metallofullerene-antibody conjugate of claim 2, wherein the antibody is selected from one or more of EGFR, HER2, HER3 and HER4 binding antibody.

5. The metallofullerene-antibody conjugate of claim 2, wherein the antibody is selected from one or more of Depatuxizumab, Varisacumab, and Trastuzumab.

6. The metallofullerene-antibody conjugate of claim 1, wherein M is selected from one or more of Holmium, Lutetium, and Scandium.

7. A pharmaceutical composition comprising the metallofullerene-antibody conjugate of claim 1 and a pharmaceutically acceptable excipient, diluent, or vehicle.

8. The pharmaceutical composition of claim 1, in a form suitable for oral administration, intravenous administration, topical administration, administration by inhalation, or administration via a suppository.

9. A method of treating a cell proliferation related disorder or disease, comprising:
administering to a patient in need thereof an effective amount of the metallofullerene-antibody conjugate of claim 1 and
administering a low-level ionizing radiation to the patient,
wherein the metallofullerene-antibody conjugate is optionally therapeutically activated by the ionizing radiation.

10. The method of claim 9, wherein Ab is a cell binding agent selected from the group comprising a chimeric, CDR-grafted, humanized, or recombinant human antibody.

11. The method of claim 10, wherein Ab is selected from the group of antibodies or antibody fragments used in cancerology, targeting: HER2, HER2, HER3, HER4 CD 52, VEGF (vascular endothelial growth factor), EGFR (epidermal growth factor receptor), CD11a, CCR4 (chemokine C-C receptor 4), CD 105, CD 123, CD 137, CD 19, CD 22, CD 23, CD 3, CD 30, CD 38, CD 4, CD 40, CD 55SC-1, CD 56, CD 6, CD 74, CD 80, CS 1 (cell-surface glycoprotein 1), CTLA4 (cytotoxic T-lymphocyte antigen 4, also known as CD152), DR5 (death receptor 5), Ep-CAM (epithelial cell adhesion molecule), folate receptor alpha, ganglioside GD2, ganglioside GD3, GPNMB, glycoprotein NMB, HGF/SF (hepatocyte growth factor/scatter factor), IGF-1 (insulin-like growth factor), IGF1-receptor (insulin-like growth factor-1 receptor), IL 13 (interleukin-13), IL 6 (interleukin-6), IL-6R (interleukin-6 receptor), immunodominant fungal antigen heat shock protein 90 (hsp90), integrin alpha 5 beta 3, MHC (major histocompatibility complex) class II, MN-antigen (also known as G250 antigen), MUC1, PD-1 (programmed death 1), PIGF (placental growth factor), PDGFRa (platelet-derived growth factor receptor alpha), prostate specific membrane antigen (PSMA), PTHrP (parathyroid hormone-related protein), CD200 receptor, receptor activator of nuclear factor kappa B ligand (RANKL), sphingosine-1-phosphate (SIP), TGF beta (transforming growth factor beta), TRAIL (tumor necrosis factor (TNF)-related apoptosis-inducing ligand) receptor 1, tumor necrosis factor receptor 2, vascular endothelial growth factor receptor 2 (VEGFR-2), CD 33, CD 20, CA125 (cancer antigen 125) or epidermal growth factor receptor.

12. The method of claim 10, wherein the antibody is selected from one or more of EGFR, HER2, HER3, and HER4 binding antibody.

13. The method of claim 9, wherein the antibody is selected from one or more of Depatuxizumab, Varisacumab, and Trastuzumab.

14. The method of claim 9, wherein M is selected from one or more of Holmium, Lutetium, and Scandium.

15. The method of claim 9, wherein the proliferative disease is cancer.

16. The method of claim 9, wherein the type of cancer is selected from one or more of lung cancer, prostate cancer, head and neck cancer, pancreatic cancer, colon/colorectal cancer, bladder cancer, thyroid cancer, breast cancer cells, liver cancer, ovarian cancer, endometrial cancer, cervical cancer cells, kidney cancer, brain cancer, and melanoma.

17. The method of claim 9, wherein the low-level ionizing radiation ranges from about 0.02 mGy to about 60 Gy.

18. The method of claim 9, wherein the metallofullerene-antibody conjugate is administered in a dose of an intravenous infusion.

19. The method of claim 9, wherein said conjugate is administered to the patient following treatment with at least one anti-cancer therapy.

20. A method of imaging a tumor in a patient, said method comprises:
administering to the patient an effective amount of endohedral metallofullerene-antibody conjugate of claim 1 and
exposing a tissue of the patient to a low-level ionizing radiation.
